(19) United States
(12) Patent Application Publication
(10) Pub. No.: US 2025/0054600 A1
(43) Pub. Date: Feb. 13, 2025

(54) DIETARY MANAGEMENT SYSTEM AND METHODS

(71) Applicant: Fresenius Medical Care Holdings Inc., Waltham, MA (US)

(72) Inventors: Peter Kotanko, New York, NY (US); Hanjie Zhang, Manhasset, NY (US); Lin-Chun Wang, New York, NY (US); Ulrike Kotanko, New York, NY (US); Nancy Ginsberg, Hartsdale, NY (US)

(73) Assignee: Fresenius Medical Care Holdings Inc., Waltham, MA (US)

(21) Appl. No.: 18/798,134

(22) Filed: Aug. 8, 2024

Related U.S. Application Data
(60) Provisional application No. 63/518,166, filed on Aug. 8, 2023.

(51) Int. Cl. G16H 20/60; G16H 10/60

(52) U.S. Cl. CPC G16H 20/60; G16H 10/60

(57) ABSTRACT

This disclosure teaches the generation of multiple recipes for patients with chronic kidney disease. The recipes are generated by application of a large language model to patient data, including both medical and preference data. The recipes are verified by a dietician and may be used to generate shopping lists and instructions for an automated food preparation device.

### CROSS-REFERENCE TO RELATED APPLICATIONS

This application claims the benefit of U.S. Provisional Patent Application Ser. No. 63/518,166, filed on Aug. 8, 2023, the disclosure of which is incorporated herein by reference.

### TECHNICAL FIELD

The present disclosure generally relates to specialized patient nutrition. Particularly, but not exclusively, the present disclosure relates to the formulation and preparation of meals for patients with chronic kidney disease.

### BACKGROUND

Chronic kidney disease (CKD) is often accompanied by specific and onerous dietary restrictions. Reduced renal function may require a diet low in sodium, potassium, and/or phosphorus. Other requirements, like necessary quantities of protein and/or fat, may also be included in a patient's diet. Comorbidities, like diabetes or hypertension, can further restrict the food choices and total menu options of patients.

To navigate these issues, patients work with trained dieticians and other medical professionals who can explain and guide eating decisions. However, practical considerations may make following the provided instructions difficult. Patients may have limited ability to shop for ingredients and prepare meals. The need to simplify instructions for patient comprehension may also limit the available options to few meal choices, which patients may eschew due to lack of variety. Certain recipes may also not fit into a patient's cultural traditions or individual tastes. In addition, the economic situation of the patient should be considered when discussing food choices.

A need exists to automate various aspects of the CKD patient's dietary process to maximize patient compliance and satisfaction with necessary medical restrictions.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure describes dietary management methods and systems that address conventional shortcomings. For example, the systems according to the present disclosure can rapidly generate a sizeable variety of recipes satisfying both medical and personal patient restrictions.

In one example, a method of dietary management comprises receiving data for a patient having chronic kidney disease (CKD), the data including patient medical data and patient preference data; querying a large language model using the received data to generate a plurality of recipes; automatically verifying the generated recipes; submitting the generated recipes to a human user for verification; and providing the generated recipes for use based on both automated and human verification of the recipes.

Alternatively, or additionally to any of the examples above, the patient medical data can include at least one nutrient maximum and at least one nutrient minimum, and wherein the generated recipes include nutrient values that satisfy the at least one nutrient maximum and the at least one nutrient minimum.

Alternatively, or additionally to any of the examples above, the method can further include generating a shopping list of ingredients for the generated recipes.

Alternatively, or additionally to any of the examples above, the shopping list can be generated by a querying a large language model using the generated recipes.

Alternatively, or additionally to any of the examples above, submitting the generated recipes to a human user can include parsing the generated recipes into a standardized format; sending the generated recipes in the standardized format to a dietary expert; and prompting the dietary expert to approve or modify the generated recipes.

Alternatively, or additionally to any of the examples above, the method can further include receiving patient feedback for the generated recipes and using the received patient feedback to query a large language model and generate a second plurality of recipes.

Alternatively, or additionally to any of the examples above, the method can further include converting each recipe from the plurality of recipes into instructions formatted for the use of an automated food preparation device and sending the formatted instructions for use in automatically preparing food.

In another example, a dietary management system includes an automated or semi-automated food preparation device; a processor; and memory comprising instructions. Which when executed by the processor, the instructions cause the system to receive data for a patient having chronic kidney disease (CKD), the data including patient medical data and patient preference data; query a large language model using the received data to generate a plurality of recipes; automatically verify the generated recipes; submit the generated recipes to a human user for verification; based on both automated and human verification of the recipes, provide the generated recipes to the automated food preparation device for use in automatically preparing food.

The instructions may alternatively or additionally cause the dietary management system to carry out any of the methods above.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a dietary management system, in accordance with embodiment(s) of the present disclosure.

FIG. 2 illustrates a process for generating recipes in accordance with embodiment(s) of the present disclosure.

FIG. 3 illustrates LLM generation of recipes from patient data in accordance with embodiments(s) of the present disclosure.

FIG. 4 illustrates a user interface for dietician recipe verification in accordance with embodiments(s) of the present disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

The devices and methods explained in the present disclosure involve the use of processing with a large language model (LLM). For purposes of this disclosure, a “large language model” is a computerized neural network of one million or more parameters, pre-trained on a data set of more than one million language tokens. One of ordinary skill will recognize a variety of LLM approaches and techniques introduced in the art, including unidirectional and bidirectional frameworks, feed-forward and feedback connections, attention mechanisms, and transformers with a variety of features. Any of these tools are recognized as large language models within the art.

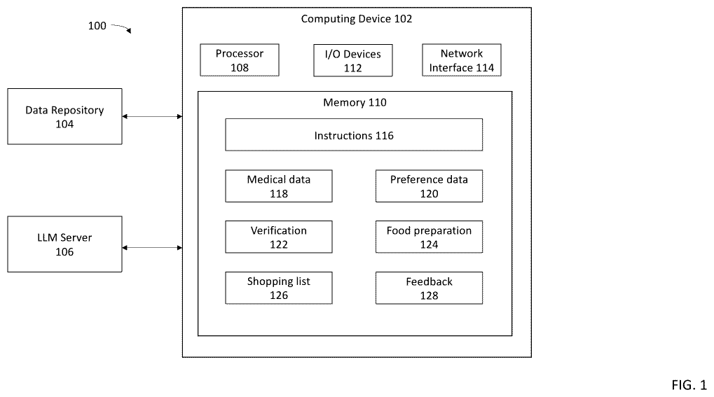

FIG. 1 illustrates a dietary management system 100, in accordance with non-limiting examples of the present disclosure. Dietary management system 100 includes a computing device 102, a data repository 104, and a LLM server 106.

The data repository 104 represents one or more systems storing data that may be accessed and provided to the computing device 102 as further described herein. Although illustrated as separate from the computing device 102, some or all the components of the data repository 104 may be components of the computer device 102.

The LLM server 106 represents a large language model (LLM) stored in one or more systems which may be local to or remote from the computing device 102. The LLM may be customized for this purpose or may be a general-purpose language model. The LLM may be cloud-based, remotely run, or locally instantiated. In some cases, the LLM may be run as part of the computer device 102. The LLM server 106 may in some cases include further filtration or processing associated with LLM input and/or input, such as tokenizing queries, formatting results, and/or recording exchanged data for later use and improvement.

The processor 108 may include circuity or processor logic, such as, for example, any of a variety of commercial processors. In some examples, processor 108 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 108 may include graphics processing portions and may include dedicated memory, multiple-threaded processing and/or some other parallel processing capability. In some examples, the processor 108 may be an application specific integrated circuit (ASIC) or a field programmable integrated circuit (FPGA).

The memory 110 may include logic, a portion of which includes arrays of integrated circuits, forming non-volatile memory to persistently store data or a combination of non-volatile memory and volatile memory. It is to be appreciated, that the memory 110 may be based on any of a variety of technologies. In particular, the arrays of integrated circuits included in memory 110 may be arranged to form one or more types of memory, such as, for example, dynamic random access memory (DRAM), NAND memory, NOR memory, or the like.

I/O devices 112 can be any of a variety of devices to receive input and/or provide output. For example, I/O devices 112 can include, a keyboard, a mouse, a joystick, a foot pedal, a display (e.g., touch, non-touch, or the like) different from display device 106, a haptic feedback device, an LED, or the like. One or more features of the endoscope may also provide input to the imaging system 100.

Network interface 114 can include logic and/or features to support a communication interface. For example, network interface 114 may include one or more interfaces that operate according to various communication protocols or standards to communicate over direct or network communication links. Direct communications may occur via use of communication protocols or standards described in one or more industry standards (including progenies and variants). For example, network interface 114 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 114 can include logic and/or features to enable communication over a variety of wired or wireless network standards (e.g., 802.11 communication standards). For example, network interface 114 may be arranged to support wired communication protocols or standards, such as, Ethernet, or the like. As another example, network interface 114 may be arranged to support wireless communication protocols or standards, such as, for example, Wi-Fi, Bluetooth, ZigBee, LTE, 5G, or the like.

Memory 110 can include instructions 116. During operation, processor 108 can execute instructions 116 to cause the computing device 102 to access data from the repository 104 associated with a particular patient. Patient data may include both patient medical data 118 and patient preference data 120, each of which may reside in records within one or more data repositories 104 or kept within memory 110.

The computing device 102 collects medical data 118 potentially relevant to a patient's dietary management. This may include patient records involving kidney function, dialysis history and planned future sessions, comorbidities and ancillary conditions, and any restrictions or requirements added by a medical professional. In some implementations, patient data may also include releases, forms, or other consent by the patient allowing or limiting the use of the patient records. For example, medical data 118 could include that an individual is diabetic, that they have one or more food allergies, that they have limited bite force due to tooth loss, or that they have reduced motor skills for handling certain utensils. In some implementations, medical data 118 may be standardized or formatted for some other purpose, such as a condition summary provided to an attending physician or nurse. One or more modules associated with the computing device 102 may filter or reformat medical data 118 as needed for use by the dietary management system 100.

The computing device 102 further collects preference data 120 potentially relevant to the patient's dietary management. This may include patient records involving demographic or cultural information as well as expressed or deduced likes, dislikes, and restrictions. Preference data 120 can also include information relevant to a patient's budget. For example, preference data 120 could include that an individual is vegetarian, that they keep kosher or halal, that they enjoy spicy food, or that they dislike pasta. Additionally, preference data 120 could include that an individual can spend less than or equal to a threshold dollar amount per day on food (e.g., $15/day, $25/day, $30/day, or the like). With some examples, preference data 120 could include a patient preferred recipe. In such an example, as will be described below, the LLM could be utilized to modify the preferred recipe to manage micronutrients (e.g., sodium, phosphorus, etc.) for a patient with CKD. In some implementations, detailed preference data 120 may be gathered by surveys filled out by a patient or on a patient's behalf. The memory 110 further includes modules associated with verification 122, food preparation 124, shopping list 126, and patient feedback 128 as further described below.

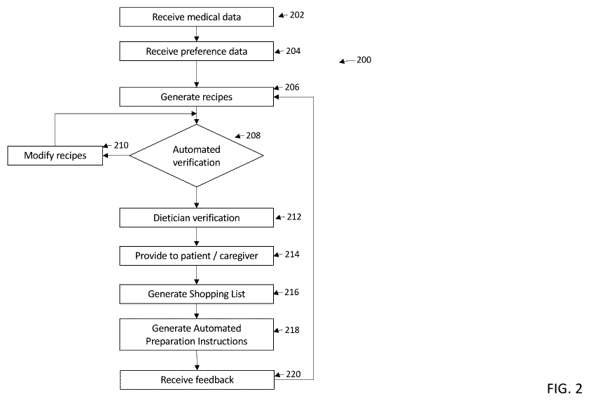

The process 200 of FIG. 2 shows steps for dietary management according to some implementations of the present disclosure. At steps 202 and 204, the computing device receives patient medical (e.g., medical data 118) and preference data (e.g., preference data 120) as described above. At step 206, a large language model (e.g., an LLM accessed via LLM server 106, or the like) is employed to generate recipes based on the patient data.

The LLM may be engaged by a variety of different approaches to generate the required recipes. In some implementations, a natural language request may be made that incorporates some or all the patient data. For example, a statement such as “Generate a set of recipes that will provide [nutritional requirements] for a patient with [medical conditions] who has [preferences]” may be submitted to the model. In some implementations, some or all the patient data may be part of the pre-training data and/or have been previously submitted to the model for learning. In some implementations, an application programming interface (API) or other communication method with the LLM server 106 may be used that can directly supply data without the use of natural language queries.

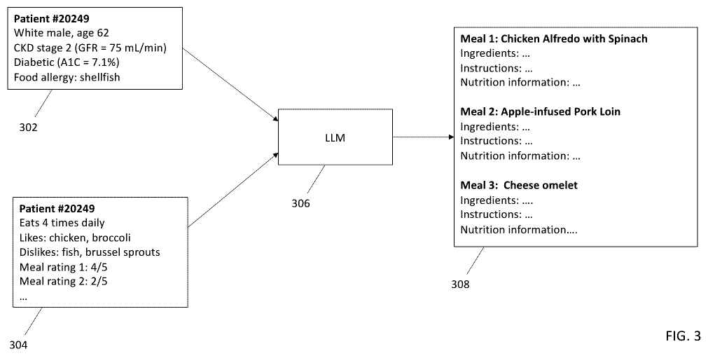

FIG. 3 illustrates an example of such recipe generation. Medical data 302 and preference data 304 are each gathered for a particular patient. In the illustrated example, the medical data 302 includes the patient's race, age, and sex, the specifics of the patient's chronic kidney disease diagnosis and stage, the patient's diabetes diagnosis and measured blood glucose level, and a known food allergy. This information may be collected by any combination of data processing, accessing of medical records, and provider questionnaires.

In the illustrated example, the preference data 304 includes the patient's eating habits, likes, dislikes, budget, preferred recipes, geographic location, and available feedback about previous meals (which may be collected as further described herein). Data may be collected by surveying the patient, by their providers and/or caretakers, and by previous interactions with their dietician.

The data 302 and 304 is submitted to the LLM 306, which generates recipes 308. Each recipe may include, for example, a list of ingredients, a list of preparation instructions, and nutrition information. The generated recipes may be presented in text format or in another data format. With some examples, data 302 and 304, which can include a preferred recipe, is submitted to the LLM 306 and the preferred recipe is modified to adjust the micronutrients to adjust the recipe to one suitable for the patient. The LLM can further be queried to provide a comparison of the original and adjusted micronutrients.

Returning to the process 200 of FIG. 2, once the recipes are generated, at step 206, the recipes are subjected to an automated verification process at step 208. In some implementations, this automated process does not include machine learning or stochastic modeling, but instead includes predefined logic to assure a variety of requirements are met, such as:
The generated recipes name only real ingredients that are readily available and edible.
The recipe instructions reflect actual food preparation steps.
Provided nutrition information accurately reflects the listed ingredients and amounts.
No unnecessary or inappropriate information is included.
The expected food costs are less than or equal to the budget.

If any of these requirements are not met, then at step 210 the recipes may be modified. Modifying the recipes may, in some implementations, involve additional use of the LLM or another LLM with further parameters provided. Certain recipe modifications may be made without the further use of LLM, such as correcting erroneous nutritional values or removing unnecessary information. The modified recipes may then be resubmitted for automated verification (e.g., step 208 may be repeated on the updated or modified recipes).

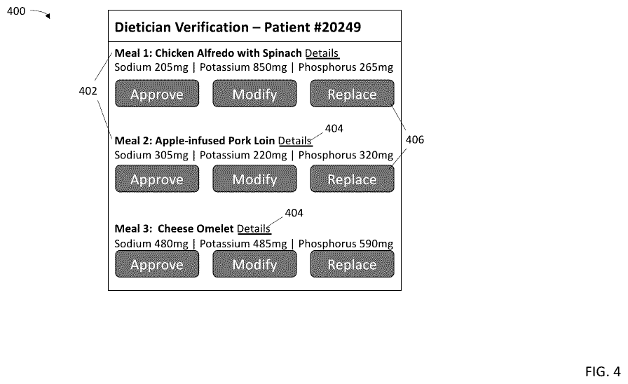

Recipes that have passed automated verification are then provided to a dietician for verification (step 212). An example of a display 400 for use by a dietician is shown in FIG. 4. Here, nutrition information for each recipe 402 is shown. The dietician can receive more information about each recipe by selecting a link 404. Each recipe may be accepted, modified, or replaced by selecting the appropriate input 406.

The final version of the recipes as modified and approved by the dietician are provided to the patient and/or the patient's caregiver (step 214). The recipes as provided in this step may be customized for easy use, and may include translation, images, text to speech, and other accessibility features as needed for convenient reference in food preparation. With some examples, the recipes may include expected food costs, locations to purchase raw food supplies, prices at such locations, etc.

At step 216, the approved recipes may also be used to create a shopping list, including some or all the ingredients across the recipes. The shopping list may include generic item names, or in some implementations, may reference specific products or brands available for sale either online or at one or more food supply businesses, such as a grocery or convenience store. In some implementations, a LLM may be used to generate the shopping list; if so, automatic and/or expert verification of the list may again be used as described above.

At step 218, the approved recipes may be used to create instructions for automatic preparation. The automated preparation device may include instructions in a particular format, including a proprietary format when necessary. In some implementations, a patient or caregiver may be able to specify a particular automated food preparation device available. One of ordinary skill will also recognize that the availability of a particular automated food preparation device may be used as input for the generation of recipes in step 206 and for automated verification in step 208, such that the recipes are appropriate for preparation using the identified device. At step 218, the instructions can be generated in a format (e.g., API) that can be downloaded or sent to the automatic food preparation device. Further, at step 218, the processor 200 can include sending (e.g., transmitting, or the like) the instructions to the food preparation device. For example, where the food preparation device is coupled to the internet, step 218 can transmit the instructions to the food preparation device.

At step 220, the system may solicit and receive feedback from a patient and/or caregiver regarding one or more of the meals. Feedback may come in the form of a numerical rating and/or a binary assessment as to whether a meal was liked. In some implementations, more detailed feedback may be provided, associated with specific dishes and/or ingredients. The feedback is then included in the patient's preference data for the generation of further meals.

Terms used herein should be accorded their ordinary meaning in the relevant arts, or the meaning indicated by their use in context, but if an express definition is provided, that meaning controls.

Herein, references to “one embodiment”, “an embodiment”, “one implementation”, or “an implementation” do not necessarily refer to the same embodiment or implementation, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A method of dietary management, comprising:
receiving data for a patient having chronic kidney disease (CKD), the data including patient medical data and patient preference data;
querying a large language model using the received data to generate a plurality of recipes;
automatically verifying the generated recipes;
submitting the generated recipes to a human user for verification; and
providing the generated recipes for use based on both automated and human verification of the recipes.

2. The method of claim 1, wherein the patient medical data includes at least one nutrient maximum and at least one nutrient minimum, and wherein the generated recipes include nutrient values that satisfy the at least one nutrient maximum and the at least one nutrient minimum.

3. The method of claim 1, further comprising:
generating a shopping list of ingredients for the generated recipes.

4. The method of claim 3, wherein the shopping list is generated by a querying a large language model using the generated recipes.

5. The method of claim 1, wherein submitting the generated recipes to a human user comprises:
parsing the generated recipes into a standardized format;
sending the generated recipes in the standardized format to a dietary expert; and
prompting the dietary expert to approve or modify the generated recipes.

6. The method of claim 1, further comprising:
receiving patient feedback for the generated recipes; and
using the received patient feedback to query a large language model and generate a second plurality of recipes.

7. The method of claim 1, further comprising:
converting each recipe from the plurality of recipes into instructions formatted for the use of an automated food preparation device; and
sending the formatted instructions for use in automatically preparing food.

8. A dietary management system, comprising:
an automated food preparation device;
a processor;
memory comprising instructions, which when executed by the processor cause the dietary management system to:
receive data for a patient having chronic kidney disease (CKD), the data including patient medical data and patient preference data;
query a large language model using the received data to generate a plurality of recipes;
automatically verify the generated recipes;
submit the generated recipes to a human user for verification;
based on both automated and human verification of the recipes, provide the generated recipes to the automated food preparation device for use in automatically preparing food.

9. The system of claim 8, wherein the instructions further cause the dietary management system to convert the generated recipes into instructions formatted for the use of the automated food preparation device

10. The system of claim 8, wherein the patient medical data includes at least one nutrient maximum and at least one nutrient minimum, and wherein the generated recipes include nutrient values that satisfy the at least one nutrient maximum and the at least one nutrient minimum.

11. The system of claim 8, wherein the instructions further cause the dietary management system to generate a shopping list of ingredients for the generated recipes.

12. The system of claim 11, wherein the shopping list is generated by a querying a large language model using the generated recipes.

13. The system of claim 8, wherein submitting the generated recipes to a human user comprises:
parsing the generated recipes into a standardized format;
sending the generated recipes in the standardized format to a dietary expert; and
prompting the dietary expert to approve, modify, or replace the generated recipes.

14. The system of claim 8, wherein the instructions further cause the dietary management system to:
receive patient feedback for the generated recipes; and
use the received patient feedback to query a large language model and generate a second plurality of recipes.

15. A computer-readable memory storage device, comprising instructions, which when executed by a processor of a dietary management system, cause the dietary management system to:
query a large language model (LLM) to generate a plurality of recipes for a patient using at least patient medical data and patient preference data as input to form the query, wherein the patient has chronic kidney disease (CKD);
receive, from the LLM, a plurality of recipes;
verify, by the processor via an automated verification process, the plurality of recipes;
submit the generated recipes to a human user for verification; and
provide the generated recipes for use based on both the automated and human verification of the recipes.

16. The computer-readable memory storage device of claim 15, wherein the patient medical data includes at least one nutrient maximum and at least one nutrient minimum, and wherein the generated recipes include nutrient values that satisfy the at least one nutrient maximum and the at least one nutrient minimum.

17. The computer-readable memory storage device of claim 15, the instructions, when executed by the processor, further cause the dietary management system to query the LLM to generate a shopping list of ingredients for the plurality of recipes.

18. The computer-readable memory storage device of claim 15, the instructions, when executed by the processor, further cause the dietary management system to:
parse the generated recipes into a standardized format;
send the generated recipes in the standardized format to a computing device associated with a dietary expert; and
prompt, via the computing device, the dietary expert to approve or modify the generated recipes.

19. The computer-readable memory storage device of claim 15, the instructions, when executed by the processor, further cause the dietary management system to:
receive patient feedback for the generated recipes; and
use the received patient feedback to query the LLM and generate a second plurality of recipes.

20. The computer-readable memory storage device of claim 15, the instructions, when executed by the processor, further cause the dietary management system to:
convert each recipe from the plurality of recipes into instructions formatted for the use of an automated food preparation device; and
send the formatted instructions for use in automatically preparing food.
